SEQUENCE LISTING (25 sequences)

SEQ ID NO: 1 - DNA, 35 bp, Artificial Sequence
  Synthetic construct
gggtaatcgcttatcctaatacgactcactatagg

SEQ ID NO: 2 - DNA, 40 bp, Artificial Sequence
  Synthetic construct
gggtaatcgcttatcccacattaatacgactcactatagg

SEQ ID NO: 3 - DNA, 45 bp, Artificial Sequence
  Synthetic construct
gggtaatcgcttatccctattatcattaatacgactcactatagg

SEQ ID NO: 4 - DNA, 50 bp, Artificial Sequence
  Synthetic construct
gggtaatcgcttatcccattattattatcattaatacgactcactatagg

SEQ ID NO: 5 - DNA, 35 bp, Artificial Sequence
  Synthetic construct
taatacgactcactatagggggtaatcgcttatcc

SEQ ID NO: 6 - DNA, 40 bp, Artificial Sequence
  Synthetic construct
taatacgactcactataggaattgtgagcggataacaatt

SEQ ID NO: 7 - DNA, 50 bp, Artificial Sequence
  Synthetic construct
attaaatgtgagcggataacattgacattgtgagcggataacaagatact

SEQ ID NO: 8 - DNA, 16 bp, Artificial Sequence
  Synthetic construct
gggtaatcgcttatcc

SEQ ID NO: 9 - protein, 1053 aa, Artificial Sequence
  Synthetic construct
ATGCCACCCAAGCGCAAAACGCTTAACGCTGCGGCTGAGGCTAACGCTCATGCCGACGGACACGCCGACGGAAACGCCGACGGACACGTGGCCAATACGGCCGCGTCCTCGAATAATGCGAGGTTCGCTGATCTCACTAACATCGATACTCCGGGTCTGGGACCCACAACTACGACCCTGCTCGTGGAACCAGCACGCTCAAAGCGTCAACGAGTGTCCCGCGCATGCGACCAGTGCCGTGCAGCCCGAGAGAAATGCGACGGAATACAGCCTGCGTGTTTCCCGTGCGTTTCCCAGGGAAGGTCCTGCACTTATCAGGCTTCGCCGAAAAAGAGGGGAGTTCAAACCGGTTATATTCGTACGCTGGAGCTCGCCCTCGCCTGGATGTTTGAAAATGTCGCGCGTTCCGAAGATGCCTTGCATAACCTCCTCGTCCGTGACGCCGGACAAGGATCAGCTCTGCTCGTTGGTAAAGATTCGCCGGCTGCCGAGCGACTCCATGCCCGTTGGGCTACTAGCCGTGTCAATAAGAGCATTACCCGCCTCCTCCGTCAGTTGGAGCTCCCTCCTACCGCCACGGCTACGGCCTCGATAATGCCGCACGTGATGGAGCAGCCTCTCAGTACCAGCATTAACCCCGTCAACGACCGCTTCAACGGTATTCCCAACCCCACTCCGTATAACTCCGATGCAGCTCTCGATGCTATCACTCAGACCAACGATTATGGAAGCGTAAATACACATGGTATCCTCTCTACTTACCCGCCACCGGCTACGCACCTTAATGAAGCTTCCGTCGCTCTCGCTCCCGGTGGCGCCCCCCCCCGACCGCCTCCTCCGTATGTTGACAGCACGACCAATCACCCGCCGTACCACTCGAATCTGGTTCCAATGGCGAACTTTGGTTACTCGACCGTTGATTACGATGCCATGGTTGACGATTTGGCTAGCATTGAATACACGGACGCTGTGGATGTCGACCCACAGTTTATGACCAATCTGGGATTCGTTCCTGGATGTAACTTCTCCGACATTAATACATACGAACAGTGA

SEQ ID NO: 10 - DNA, 17 bp, Artificial Sequence
  Synthetic construct
taatacgactcactata

SEQ ID NO: 11 - DNA, 21 bp, Artificial Sequence
  lacO site
aattgtgagcggataacaatt

SEQ ID NO: 12 - DNA, 19 bp, Artificial Sequence
  Synthetic construct
tccctatcagtgatagaga

SEQ ID NO: 13 - DNA, 33 bp, Artificial Sequence
  Synthetic construct
gggtaatcgcttatcctaatacgactcactata

SEQ ID NO: 14 - DNA, 35 bp, Artificial Sequence; identical to SEQ ID NO: 5
  Synthetic construct

SEQ ID NO: 15 - DNA, 42 bp, Artificial Sequence
  Synthetic construct
taatacgactcactataggggaattgtgagcggataacaatt

SEQ ID NO: 16 - DNA, 49 bp, Artificial Sequence
  Synthetic construct
ataaatgtgagcggataacattgacattgtgagcggataacaagatact

SEQ ID NO: 17 - DNA, 54 bp, Artificial Sequence
  Synthetic construct
gggtaatcgcttatcctaatacgactcactataggtccctatcagtgatagaga

SEQ ID NO: 18 - DNA, 40 bp, Artificial Sequence; identical to SEQ ID NO: 2
  Synethtic construct

SEQ ID NO: 19 - DNA, 45 bp, Artificial Sequence; identical to SEQ ID NO: 3
  Synthetic construct

SEQ ID NO: 20 - DNA, 50 bp, Artificial Sequence; identical to SEQ ID NO: 4
  Synthetic construct

SEQ ID NO: 21 - DNA, 40 bp, Artificial Sequence
  Synthetic construct
taatacgactcactatagggtgtagggtaatcgcttatcc

SEQ ID NO: 22 - DNA, 45 bp, Artificial Sequence
  Synethtic construct
taatacgactcactataggctattatcatgggtaatcgcttatcc

SEQ ID NO: 23 - DNA, 50 bp, Artificial Sequence
  Synthetic construct
taatacgactcactataggcattattattatcatgggtaatcgcttatcc

SEQ ID NO: 24 - DNA, 67 bp, Artificial Sequence
  Synthetic construct
taatacgactcactataggggaattgtgagcggataacaattcccgactagaaataattttgtttaa

SEQ ID NO: 25 - DNA, 11 bp, Artificial Sequence
  Synthetic construct
gaggatccatg

(19) United States
(12) Patent Application Publication
(10) Pub. No.: US 2024/0026368 A1
(43) Pub. Date: Jan. 25, 2024

(54) SYSTEMS AND METHODS FOR CONTROLLING BACTERIAL TRANSCRIPTION

(71) Applicant: UNIVERSITY OF UTAH RESEARCH FOUNDATION, Salt Lake City, UT (US)

(72) Inventors: Tara Deans, Salt Lake City, UT (US); Ian Cody MacDonald, Salt Lake City, UT (US)

(21) Appl. No.: 18/043,792

(22) PCT Filed: Sep. 2, 2021
(86) PCT No.: PCT/US2021/048947

§ 371 Date: Mar. 2, 2023

Related U.S. Application Data
(60) Provisional application No. 63/073,554, filed on Sep. 2, 2020.

(51) Int. Cl. C12N 15/72

(52) U.S. Cl. CPC C12N 15/72

(57) ABSTRACT

Disclosed herein, are methods of controlling expression of one or more genes of interest in one or more bacterial cells using a eukaryotic transcription factor, and inducible binary expression systems thereof.

### CROSS REFERENCE TO RELATED APPLICATIONS

This application claims the benefit of the filing date of U.S. Provisional Application 63/073,554, which was filed on Sep. 2, 2020. The content of this earlier filed application is hereby incorporated by reference herein in its entirety.

### INCORPORATION OF THE SEQUENCE LISTING

The present application contains a sequence listing that was submitted in ASCII format via EFS-Web concurrent with the filing of the application, containing the file name 21101_0381P1_Sequence_Listing which is 16,384 bytes in size, created on Sep. 2, 2021, and is herein incorporated by reference in its entirety.

### BACKGROUND

The field of synthetic biology has revolutionized how cells can be reprogrammed with the assembly of genetic parts into more complex gene circuits. This bottom-up approach can be applied to many organisms and provides a means to control cell behavior by engineering gene circuits to program cells with novel gene expression patterns such as switches (A. L. Chang, et al., Current opinion in biotechnology 23, 679-688 (2012); T. L. Deans, et al. Cell 130, 363-372 (2007); M. Fitzgerald, et al., ACS synthetic biology 6, 2014-2020 (2017); T. S. Gardner, et al., Nature 403, 339-342 (2000); A. A. Green, et al., Cell 159, 925-939 (2014); B. P. Kramer et al., Nature biotechnology 22, 867-870 (2004); and L. R. Polstein, et al., ACS synthetic biology 6, 2003-2013 (2017), oscillators (A. Aulehla, O. Pourquie, Current opinion in cell biology 20, 632-637 (2008); T. Danino, et al., Nature 463, 326-330 (2010); M. B. Elowitz, S. Leibler, Nature 403, 335-338 (2000); E. Fung et al., Nature 435, 118-122 (2005); J. Stricker et al., Nature 456, 516-519 (2008); and M. Tigges, et al., Nature 457, 309-312 (2009)), logic gates (C. C. Guet, et al., Science 296, 1466-1470 (2002); S. Mukherji, A. van Oudenaarden, Nature reviews. Genetics 10, 859-871 (2009); P. Siuti, et al., Nature biotechnology 31, 448-452 (2013); T. S. Moon, et al., Nature 491, 249-253 (2012); and C. A. Voigt, Current opinion in biotechnology 17, 548-557 (2006)), and enhanced protein production (T. Ellis, et al., Nature biotechnology 27, 465-471(2009); T. K. Lu, et al., Nature biotechnology 27, 1139-1150 (2009); A. S. Khalil, J. J. Collins, Nature reviews. Genetics 11, 367-379 (2010); and K. Temme, et al., Nucleic acids research 40, 8773-8781 (2012)). Genetic parts from diverse organisms are used to establish orthogonal, or independent, functions within cells. The control elements for regulating orthogonal genetic circuits do not exist in the host, therefore avoiding cross-talk in engineered cells (K. Temme, et al., Nucleic acids research 40, 8773-8781 (2012); and B. A. Blount, et al., PloS one 7, e33279 (2012)). Traditionally, genetic parts from simpler prokaryotic organisms are moved to more complex eukaryotic cells to provide orthogonal genetic control in these higher organisms (C. J. Potter, et al., Cell 141, 536-548 (2010); T. L. Deans, ACM Journal on Emerging Technologies in computing systems (JETC) 11, 21:21-21:11 (2014); I. C. MacDonald, T. L. Deans, Adv Drug Deliv Rev 105, 20-34 (2016); M. S. Weisenberger, T. L. Deans, J Ind Microbiol Biotechnol 45, 599-614 (2018); S. Auslander, et al., Angew Chem Int Ed Engl 56, 6396-6419 (2017); J. J. Collins et al., Nature 509, 155-157 (2014); and A. S. Khalil et al., Cell 150, 647-658 (2012)).

### SUMMARY

Disclosed herein are methods of controlling expression of one or more genes of interest in one or more bacterial cells, the method comprising: a) transforming the cells with a first construct, wherein the first construct comprises a first prokaryotic promoter operably linked to a eukaryotic transcription factor, and b) transforming the cells with a second construct, wherein the second construct comprises a second prokaryotic promoter operably linked to a eukaryotic transcription factor binding site sequence and one or more genes of interest, wherein the eukaryotic transcription factor binding site sequence and the eukaryotic transcription factor are a binding pair, whereby expression of the one or more genes of interest in said one or more bacterial cells is regulated by binding of said eukaryotic transcription factor to the eukaryotic transcription factor binding site sequence.

Disclosed herein are methods of controlling expression of one or more genes of interest in one or more bacterial cells, the method comprising: transforming the cells with a first construct, wherein the first construct comprises a first prokaryotic promoter operably linked to a eukaryotic transcription factor, and wherein said cells comprise a second construct, wherein the second construct comprises a second prokaryotic promoter operably linked to a eukaryotic transcription factor binding site sequence and one or more genes of interest, wherein the eukaryotic transcription factor binding site sequence and the eukaryotic transcription factor are a binding pair, whereby expression of the one or more genes of interest in said one or more bacterial cells is regulated by binding of said eukaryotic transcription factor to the eukaryotic transcription factor binding site sequence.

Disclosed herein are methods of controlling expression of one or more genes of interest in one or more bacterial cells, the method comprising: transforming the cells with a second construct, wherein the second construct comprises a second prokaryotic promoter operably linked to a eukaryotic transcription factor binding site sequence and a gene of interest, wherein the eukaryotic transcription factor binding site sequence and the eukaryotic transcription factor are a binding pair, and wherein said cells comprise a first construct, wherein the first construct comprises a first prokaryotic promoter operably linked to a eukaryotic transcription factor, whereby expression of the one or more genes of interest in said one or more bacterial cells is regulated by binding of said eukaryotic transcription factor to the eukaryotic transcription factor binding site sequence.

Disclosed herein are inducible binary expression systems, the systems comprising: a) a first and second prokaryotic promoter; b) an operon, wherein the operon is endogenous to a prokaryotic cell; c) a eukaryotic transcription factor, wherein the first prokaryotic promoter is operably linked to the eukaryotic transcription factor; d) a eukaryotic transcription factor binding site sequence, wherein the second prokaryotic promoter is operably linked to the eukaryotic transcription factor binding site sequence and wherein the eukaryotic transcription factor binding site sequence and the eukaryotic transcription factor are a binding pair; and e) one or more genes of interest, wherein the one or more genes of interest is regulated by the binding of the eukaryotic transcription factor to the eukaryotic transcription factor binding site sequence.

Disclosed herein are inducible binary expression systems, the systems comprising: a prokaryotic cell comprising: a) a first and second prokaryotic promoter; b) an operon, wherein the operon is endogenous to a prokaryotic cell; c) an eukaryotic transcription factor, wherein the first prokaryotic promoter is operably linked to the eukaryotic transcription factor; d) a eukaryotic transcription factor binding site sequence, wherein the second prokaryotic promoter is operably linked to the eukaryotic transcription factor binding site sequence and wherein the eukaryotic transcription factor binding site sequence binds to the eukaryotic transcription factor; and e) one or more genes of interest, wherein the one or more genes of interest is regulated by the binding of the eukaryotic transcription factor to the eukaryotic transcription factor binding site sequence.

Disclosed herein are prokaryotic cells comprising a prokaryotic promoter operably linked to a eukaryotic transcription factor.

Disclosed herein are prokaryotic cells comprising a prokaryotic promoter operably linked to a eukaryotic transcription factor binding site sequence.

Disclosed herein are prokaryotic cells comprising a first prokaryotic promoter operably linked to a eukaryotic transcription factor and a second prokaryotic promoter operably linked to a eukaryotic transcription factor binding site sequence.

Disclosed herein are prokaryotic cells comprising: a first prokaryotic promoter operably linked to a eukaryotic transcription factor, and wherein the prokaryotic cell comprises a construct, wherein the construct comprises a second prokaryotic promoter operably linked to a eukaryotic transcription factor binding site sequence and one or more genes of interest.

Disclosed herein are prokaryotic cells comprising a second prokaryotic promoter operably linked to a eukaryotic transcription factor binding site sequence and a gene of interest, and wherein the prokaryotic cell comprises a construct, wherein the construct comprises a first prokaryotic promoter operably linked to a eukaryotic transcription factor.

Disclosed herein are prokaryotic cells comprising: a) a first construct, wherein the first construct comprises a first prokaryotic promoter operably linked to a eukaryotic transcription factor, and b) a second construct, wherein the second construct comprises a second prokaryotic promoter operably linked to a eukaryotic transcription factor binding site sequence and one or more genes of interest.

### BRIEF DESCRIPTION OF THE DRAWINGS

FIGS. 1A-C show constructs for use in the systems and methods disclosed herein in bacteria. FIG. 1A is a schematic of a control plasmid with the bacteriophage T7 promoter constitutively expressing GFP followed by a T7 terminator sequence (T) (top). FIG. 1A also shows a schematic of placing QUAS (orange square with lines) directly upstream of the T7 promoter (QUAS-0-T7) driving the expression of GFP (green) (middle). FIG. 1A also shows a schematic of adding the constitutive expression of QF (orange) to the system (QUAS-0-T7+QF) (bottom). When QF is present it binds to QUAS and activates the expression of GFP (green). FIG. 1A also shows flow cytometry quantifying the GFP fluorescence over a four-hour period after the initial induction of 0.5 mM IPTG (added at time zero). A two-tailed t-test was performed to determine statistical significance (P<0.01) between the T7 control and components of the construct upstream of the T7 promoter. An asterisk (*) represents statistical significance. FIG. 1B is a schematic of QUAS placed directly downstream of the T7 promoter (T7-0-QUAS) driving the expression of GFP (top). FIG. 1A shows a schematic of adding the constitutive expression of QF to the system (bottom). When QF is present it binds to QUAS and activates the expression of GFP (T7-0-QUAS+QF). FIG. 1A shows flow cytometry quantifying the GFP fluorescence over a four hour period after the initial induction of 0.5 mM IPTG (added at time zero) to initiate the transcription of T7RNAP to be available for transcribing genes downstream of the T7 promoters. A two-tailed t-test was performed to determine statistical significance (P<0.01) between the T7 control and components of the system downstream of the T7 promoter. An aster (*) represents statistical significance. FIG. 1C is a schematic controlling ccdB expression by replacing GFP with the toxin ccdB (turquoise) with the QUAS directly upstream of the T7 promoter (top) and adding the constitutive expression of QF (orange) to the system (bottom). When QF is present it binds to QUAS and activates the expression of the ccdB toxin. Percent colony forming units per milliliter (% CFU/mL) over a four hour period after induction of 0.5 mM IPTG (added at time zero) to initiate the transcription of T7RNAP in the absence of QF (grey line) and the presence of QF (blue line). In the experiments, IPTG was added to initiate transcription of T7RNAP, allowing transcription of T7-controlled genes. Each experiment consisted of generating data from at least three separate bacterial colonies grown in overnight cultures, where circles represent individual data points in the plots. These experiments were repeated independently at least three times with similar results. The geometric mean of each sample was calculated via FlowJo, and error bars indicate standard deviation. Fluorescence values were normalized to the T7 control expression at 1 hour after IPTG induction. The error bars indicate 95% confidence intervals of the mean of fluorescence, and data are presented as mean±standard deviation.

FIGS. 2A-C show constructs and QUAS spacing upstream of the T7 promoter. FIG. 2A is a schematic depicting QUAS (orange square with lines) spaced 5 base pairs upstream of the T7 promoter (QUAS-5-T7) driving the expression of GFP (green) (top) and the effect of QF (orange) on GFP expression (bottom). In the absence of QF, the transcription of GFP is not initiated. When QF is present, (QUAS-5-T7+QF) it binds to QUAS and activates the transcription of GFP. Flow cytometry quantifying GFP fluorescence over a ten-hour period after the initial induction of 0.5 mM IPTG (added at time zero) to initiate the transcription of T7RNAP without QUAS (purple), with QUAS 5 base pairs upstream of the T7 promoter in the absence (blue), and presence (green) of QF. A two-tailed t-test was performed to determine statistical significance (P<0.02) between the T7 control and components of the system with QUAS placed 5 base pairs upstream of the T7 promoter. An asterisk (*) represents statistical significance. FIG. 2B is a schematic depicting QUAS (orange square with lines) spaced 10 base pairs upstream of the T7 promoter (QUAS-10-T7) driving the expression of GFP (green) (top) and the effect of QF (orange) on GFP expression (bottom). In the absence of QF the transcription of GFP is not initiated, however, in the presence of QF (QUAS-10-TF+QF) it binds to QUAS and activates the transcription of GFP. Flow cytometry quantifying GFP fluorescence over a ten-hour period after the initial induction of 0.5 mM IPTG (added at time zero) to initiate the transcription of T7RNAP without QUAS (purple), with QUAS 10 base pairs upstream of the T7 promoter in the absence (blue) and presence (green) of QF. A two-tailed t-test was performed to determine statistical significance (P<0.002) between the T7 control and components of the system with QUAS placed 10 base pairs upstream of the T7 promoter. An asterisk (*) represents statistical significance. FIG. 2C is a schematic depicting QUAS (orange square with lines) spaced 15 base pairs upstream of the T7 promoter (QUAS-15-T7) driving the expression of GFP (green) (top) and the effect of QF (orange) on GFP expression (bottom). In the absence of QF the transcription of GFP is not initiated. When QF is present, (QUAS-15-T7+QF) it binds to QUAS and activates the transcription of GFP. Flow cytometry quantifying GFP fluorescence over a ten-hour period after the initial induction of 0.5 mM IPTG (added at time zero) to initiate the transcription of T7RNAP without QUAS (purple), with QUAS 15 base pairs upstream of the T7 promoter in the absence (blue), and presence (green) of QF. A two-tailed t-test was performed to determine statistical significance (P<0.007) between the T7 control and components of the system with QUAS placed 15 base pairs upstream of the T7 promoter. An asterisk (*) represents statistical significance. Each experiment consisted of generating data from at least three separate bacterial colonies grown in overnight cultures, where circles represent individual data points in the plots. These experiments were repeated independently at least three times with similar results. The geometric mean of each sample was calculated via FlowJo, and error bars indicate standard deviation. Fluorescence values were normalized to the T7 control (purple) expression at 1 hour. The error bars indicate 95% confidence intervals of the mean of fluorescence, and data are presented as mean±standard deviation.

FIGS. 3A-C show an example of constructs that can be used in the systems and methods disclosed herein. QUAS spacing downstream of the T7 promoter. FIG. 3A is a schematic depicting the QUAS (orange square with lines) spaced 5 base pairs downstream of the T7 promoter (T7-5-QUAS) driving the expression of GFP (green) (top) and the effect of QF (orange) on GFP expression (bottom). In the absence of QF the transcription of GFP is not initiated. When QF if present (T7-5-QUAS+QF) it binds to QUAS and activates the transcription of GFP. Flow cytometry quantifying GFP fluorescence over a ten-hour period after the initial induction of 0.5 mM IPTG (added at time zero) to initiate the transcription of T7RNAP without QUAS (purple), with QUAS 5 base pairs downstream of the T7 promoter in the absence (blue), and presence (green) of QF. A two-tailed t-test was performed to determine statistical significance (P<0.04) between the T7 control and components of the Q system with QUAS placed 5 base pairs downstream of the T7 promoter. An asterisk (*) represents statistical significance. FIG. 3B is a schematic depicting the QUAS (orange square with lines) spaced 10 base pairs downstream of the T7 promoter (T7-10-QUAS) driving the expression of GFP (green) (top) and the effect of QF (orange) on GFP expression (bottom). In the absence of QF the transcription of GFP is not initiated. When QF if present (T7-10-QUAS+QF) it binds to QUAS and activates the transcription of GFP. Flow cytometry quantifying GFP fluorescence over a ten-hour period after the initial induction of 0.5 mM IPTG (added at time zero) to initiate the transcription of T7RNAP without QUAS (purple), with QUAS 5 base pairs downstream of the T7 promoter in the absence (blue), and presence (green) of QF. A two-tailed t-test was performed to determine statistical significance (P<0.04) between the T7 control and components of the system with QUAS placed 5 base pairs downstream of the T7 promoter. An asterisk (*) represents statistical significance. FIG. 3B is a schematic depicting QUAS (orange square with lines) spaced 10 base pairs upstream of the T7 promoter (QUAS-10-T7) driving the expression of GFP (green) (top) and the effect of QF (orange) on GFP expression (bottom). In the absence of QF the transcription of GFP is not initiated, however, in the presence of QF (QUAS-10-TF+QF) it binds to QUAS and activates the transcription of GFP. Flow cytometry quantifying GFP fluorescence over a ten-hour period after the initial induction of 0.5 mM IPTG (added at time zero) to initiate the transcription of T7RNAP without QUAS (purple), with QUAS 10 base pairs upstream of the T7 promoter in the absence (blue) and presence (green) of QF. A two-tailed t-test was performed to determine statistical significance (P<0.05) between the T7 control and components of the Q system with QUAS placed 10 base pairs upstream of the T7 promoter. An aster (*) represents statistical significance. FIG. 3C is a schematic depicting the QUAS (orange square with lines) spaced 15 base pairs downstream of the T7 promoter (T7-15-QUAS) driving the expression of GFP (green) (top) and the effect of QF (orange) on GFP expression (bottom). In the absence of QF the transcription of GFP is not initiated. When QF if present (T7-15-QUAS+QF) it binds to QUAS and activates the transcription of GFP. Flow cytometry quantifying GFP fluorescence over a ten-hour period after the initial induction of 0.5 mM IPTG (added at time zero) to initiate the transcription of T7RNAP without QUAS (purple), with QUAS 15 base pairs downstream of the T7 promoter in the absence (blue), and presence (green) of QF. A two-tailed t-test was performed to determine statistical significance (P<0.03) between the T7 control and components of the Q system with QUAS placed 5 base pairs downstream of the T7 promoter. An asterisk (*) represents statistical significance. Each experiment consisted of generating data from at least three separate bacterial colonies grown in overnight cultures, where circles represent individual data points in the plots. These experiments were repeated independently at least three times with similar results. The geometric mean of each sample was calculated via FlowJo, and error bars indicate standard deviation. Fluorescence values were normalized to the T7 control (purple) expression at 1 hour. The error bars indicate 95% confidence intervals of the mean of fluorescence, and data are presented as mean±standard deviation.

FIGS. 4A-D show constructs built with components from a system coupled with the TetR system. FIG. 4A shows a biological sensor with QUAS (orange box with lines) placed 5 base pairs upstream of the T7 promoter driving the expression of GFP (green). A TetO site (blue square with lines) was placed upstream of GFP and QF. The tetR gene is constitutively expressed by a T7 promoter (blue arrow). FIG. 4A shows a schematic in the absence of aTc (top) indicates that both the expression of QF (orange rectangle) and GFP (green rectangle) is repressed by TetR proteins since T7RNAP cannot bind. Schematic when aTc is added to the system (bottom), the TetR proteins no longer prevent T7RNAP from binding, and transcription of QF and GFP turns on. Flow cytometry quantifying the amount of GFP expression with varying amounts of aTc. FIG. 4B shows a biological sensor with QUAS (orange box with lines) placed 10 base pairs upstream of the T7 promoter driving the expression of GFP (green). The tetR gene (blue) is constitutively expressed by a T7 promoter (blue). Schematic in the absence of aTc indicates that both the expression of QF and GFP is repressed by TetR proteins (top). Schematic when aTc is added to the system, the TetR proteins no longer prevent T7RNAP from binding, and transcription of QF and GFP turns on (bottom). Flow cytometry quantifying the amount of GFP expression with varying amounts of aTc. FIG. 4C shows a low pass sensor. In this design, the T7 terminator sequence between the QF and tetR genes was removed to produce more TetR proteins. QUAS was placed directly upstream of the T7 promoter driving GFP expression. A TetO site is located upstream of both the GFP and tetR genes so they can be regulated by aTc. Schematic of the low pass sensor in the absence of aTc (top). Schematic of the low pass filter in the presence of aTc (bottom). Flow cytometry quantifying the amount of GFP expression with varying amounts of aTc. FIG. 4D shows a genetic device to produce large quantities of protein. This genetic circuit has QUAS placed directly upstream of the T7 promoter driving the expression of GFP. A TetO site was placed upstream of GFP to regulate its expression with aTc. In this circuit the QF is constitutively expressed and binds to QUAS. Schematic in the absence of aTc, TetR binds to the TetO sites to repress the expression of GFP (top). Schematic when aTc is added to the system, the TetR proteins no longer bind, enabling the T7RNAP to bind and transcribe GFP (bottom). Flow cytometry quantifying GFP expression with varying amounts of aTc. In the experiments, the dotted line indicates the maximum expression of the traditional TetR system without the system (˜3700 arbitrary units). GFP was quantified for the constructs using flow cytometry over a 10-hour period after the initial induction of 0.5 mM IPTG (added at time zero to initiate transcription of T7RNAP, allowing transcription of T7-controlled genes. Each experiment consisted of generating data from at least three separate bacterial colonies grown in overnight cultures, where circles represent individual data points in the plots. These experiments were repeated independently at least three times with similar results. A two-tailed t-test was performed to determine statistical significance (P<0.05) between the 0 ng/mL of aTc with each induction amount over 10 hours. An aster (*) represents statistical significance. The geometric mean of each sample was calculated via FlowJo, and error bars indicate standard deviation. The error bars indicate 95% confidence intervals of the mean of fluorescence, and data are presented as mean±standard deviation.

FIG. 5 shows synthesized DNA parts for placement of QUAS relative to the T7 promoter. QUAS-0-T7 (SEQ ID NO: 13) has one QUAS site (SEQ ID NO: 8) directly upstream of the T7 promoter (SEQ ID NO:10). QUAS-0-T7TetO adds a TetO (SEQ ID NO: 12) site two base pairs downstream of the promoter. QUAS was moved −5, −10, and −15 base pairs upstream and downstream of the T7 promoter. T7-0-QUAS (SEQ ID NO: 14) has one QUAS site two base pairs downstream of the T7 promoter. This is the control referred to as T7 in other figures. pLacO is an example of an engineered promoter that utilizes the native RNAP machinery. The −10 (Pribnow box) and −35 hexamer sites are named according to the number of base pairs upstream of the transcriptional start site. Endogenous RNAP binds to promoter sequence at the −10 and −35 sites. The two LacO sites (grey) allow for LacI binding and repression of the promoter. In each case, the ribosome binding site (blue RBS) and GFP (green ATG) are located downstream of the promoter. The expression of the tetR (pink ATG) gene is regulated by the binding of Lad repressor proteins. T7lacO corresponds to SEQ ID NO: 15; pLacO corresponds to SEQ ID NO: 16; QUAS-0-T7TetO corresponds to SEQ ID NO: 17; QUAS-5-T7 corresponds to SEQ ID NO: 18; QUAS-10-T7 corresponds to SEQ ID NO: 19; QUAS-15-T7 corresponds to SEQ ID NO: 20; T7-5-QUAS corresponds to SEQ ID NO: 21; T7-10-QUAS corresponds to SEQ ID NO: 22; T7-15-QUAS corresponds to SEQ ID NO: 23; TAATACGACTCACTATAGGGGAATTGTGAGCGGATAACAATTCCCGACTAGAAATAATTTTGTTTAA corresponds to SEQ ID NO: 24 and GAGGATCCATG corresponds to SEQ ID NO: 25.

FIG. 6 shows T7 expression and untransformed BL21 DE3 cell lines. GFP expression from T7 (blue), QUAS-0-T7 (orange), QUAS-0-T7+QF (yellow), and untransformed BL21 (purple) after adding 0.5 mM IPTG, and plotted on a log scale. Each experiment consisted of generating data from at least three separate bacterial colonies grown in overnight cultures, where circles represent individual data points in the plots. These experiments were repeated independently at least three times with similar results. The geometric mean of each sample was calculated via FlowJo, and error bars indicate standard deviation. A two-tailed t-test was performed to determine statistical significance (P<0.004) between the T7 control and components of the system with QUAS placed 5 base pairs downstream of the T7 promoter. An aster (*) represents statistical significance. The error bars indicate 95% confidence intervals of the mean of fluorescence, and data are presented as mean±standard deviation.

FIG. 7 shows CcdB induced death including uninduced controls. QUAS-0-T7 driving ccdB expression without IPTG (orange line), QUAS-0-T7 driving ccdB expression induced with 10 uM IPTG (black line), QUAS-0-T7 driving ccdB co-transformed with a plasmid constitutively expressing QF without IPTG (yellow line), and QUAS-0-T7 driving ccdB co-transformed with QF expression plasmid induced with 10uM IPTG (blue line). Each experiment consisted of generating data from at least three separate bacterial colonies grown in overnight cultures, where circles represent individual data points in the plots. These experiments were repeated independently at least three times with similar results. The geometric mean of each sample was calculated via FlowJo, and error bars indicate standard deviation. The error bars indicate 95% confidence intervals of the mean of fluorescence, and data are presented as mean±standard deviation.

FIG. 8 shows overlay histograms of GFP fluorescence from T7 promoters. Flow cytometry histograms include BL21 (grey) as an untransformed control without exogeneous DNA, T7 constitutively expressing GFP (pink) and T7-0-QUAS+QF (green). Hours represent time post IPTG induction of T7RNAP.

FIG. 9 shows a traditional TetR system. Schematic in the absence of aTc (top). The TetR proteins bind to the TetO site and prevent T7RNAP from binding. Schematic of the presence of aTc, which removes the TetR proteins from operator sites, allowing T7RNAP to bind and transcribe GFP (bottom). Flow cytometry quantifying GFP fluorescence at various aTc concentrations for all constructs using flow cytometry over a 10 hour period. The induction of 0.5 mM IPTG initiates the transcription of T7RNAP (added at time zero). Each experiment consisted of generating data from at least three separate bacterial colonies grown in overnight cultures, where circles represent individual data points in the plots. These experiments were repeated independently at least three times with similar results. The geometric mean of each sample was calculated via FlowJo, and error bars indicate standard deviation. A two-tailed t-test was performed to determine statistical significance (P<0.05) between the uninduced control with increasing concentrations of aTc. The error bars indicate 95% confidence intervals of the mean of fluorescence, and data are presented as mean±standard deviation. An asterix (*) represents statistical significance.

FIGS. 10A-E show growth curves of BL21 E. coli. FIG. 10A shows growth curves of bacteria transformed with plasmids containing QUAS upstream at −15 (blue), −10 (yellow), or −5 (green) nucleotides from the T7 promoter. No plasmid control (red) is BL21(D3) not transformed with any plasmids. FIG. 10B shows growth curves of bacteria transformed with plasmids containing QUAS downstream at +15 (blue), +10 (yellow), or +5 (green) nucleotides from the T7 promoter. No plasmid control (red) is BL21(D3) not transformed with any plasmids. FIG. 10C shows growth curves of bacteria co-transformed with plasmids containing QUAS upstream at −15+QF (blue), −10+QF (yellow), or −5+QF (green) nucleotides from the T7 promoter. No plasmid control (red) is bacteria not transformed with any plasmids. FIG. 10D shows growth curves of bacteria transformed with plasmids containing QUAS downstream at +15+QF (blue), +10+QF (yellow), or +5+QF (green) nucleotides from the T7 promoter. No plasmid control (red) is bacteria not transformed with any plasmids. FIG. 10E shows growth curves of bacteria transformed with the high-pass filter (orange) and compared to a T7-GFP control (green), and QUAS+10 nucleotides (yellow) and +15 nucleotides+QF (blue). Each experiment consisted of generating data from at least three separate bacterial colonies grown in overnight cultures, where circles represent individual data points in the plots. These experiments were repeated independently at least three times with similar results. The geometric mean of each sample was calculated via FlowJo, and error bars indicate standard deviation. The error bars indicate 95% confidence intervals of the mean of fluorescence, and data are presented as mean±standard deviation.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “transcription factor” refers to modular proteins that utilize distinct domains (e.g., eukaryotic transcription factor binding site) for transcriptional activation (or repression) and DNA binding. Transcription factors bind to a specific DNA sequence, referred to as the transcription binding site. Transcription factors also comprise at least one DNA-binding domain that attaches to a specific sequence of DNA adjacent to the genes that they regulate. Transcription factors can be classified based on their DNA-binding domains. The number of transcription factors found within an organism increases with genome size such that larger genomes tend to have more transcription factors per gene. For example, it is estimated that there are 2,600 proteins in the human genome that contain DNA-binding domains, and most of these are presumed to function as transcription factors (Babu M M, et al. (June 2004) Current Opinion in Structural Biology. 14 (3): 283-91).

As used herein, “eukaryotic transcription factor” refers to transcription factor originating or associated with a eukaryotic system. Classes of eukaryotic transcription factors include but are not limited to mechanism of action, regulatory function, and sequence homology in their DNA-binding domains. Examples of eukaryotic transcription factors include but are not limited to SP1, AP-1, C/EBP, heat shock factor, ATF/CREB, cMyc, Oct-1, NF-1 and QF. Eukaryotic transcription is a process that eukaryotic cells use to copy genetic information stored in DNA into units of transportable complementary RNA replica. Gene transcription occurs in both eukaryotic cells and prokaryotic cells. In a prokaryotic cell, the RNA polymerase initiates the transcription of different types of RNA. Both transcription and translation take place in the cytoplasm of the prokaryotic cell. In a eukaryotic cell, the RNA polymerase comes in three variations, each translating a different type of gene. Eukaryotic cells have a nucleus that separates the transcription from translation. Eukaryotic transcription proceeds in three sequential stages: initiation, elongation, and termination.

As used herein, “eukaryotic transcription factor binding site” refers to a DNA binding site or a eukaryotic binding sequence in which the eukaryotic transcription factor binds. The eukaryotic transcription factor binding site and the eukaryotic transcription factor are binding partners and together make a binding pair (e.g., QF/QUAS). For example, the eukaryotic transcription factor (e.g., QF) bind to the eukaryotic transcription factor binding site (e.g., QUAS). The sequence of QUAS is: GGGTAATCGCTTATCC (SEQ ID NO: 8).

As used herein, the term “QF”, relates to QA-1F, a regulatory gene from the Neurospora crassa qa gene cluster, which is a transcriptional activator that binds to a 16-base pair sequence present in one or more copies upstream of each qa gene. The sequence of QF is:

(SEQ ID NO: 9)
ATGCCACCCAAGCGCAAAACGCTTAACGCTGCGGCTGAGGCTAACGCTC
ATGCCGACGGACACGCCGACGGAAACGCCGACGGACACGTGGCCAATAC
GGCCGCGTCCTCGAATAATGCGAGGTTCGCTGATCTCACTAACATCGAT
ACTCCGGGTCTGGGACCCACAACTACGACCCTGCTCGTGGAACCAGCAC
GCTCAAAGCGTCAACGAGTGTCCCGCGCATGCGACCAGTGCCGTGCAGC
CCGAGAGAAATGCGACGGAATACAGCCTGCGTGTTTCCCGTGCGTTTCC
CAGGGAAGGTCCTGCACTTATCAGGCTTCGCCGAAAAAGAGGGGAGTTC
AAACCGGTTATATTCGTACGCTGGAGCTCGCCCTCGCCTGGATGTTTGA
AAATGTCGCGCGTTCCGAAGATGCCTTGCATAACCTCCTCGTCCGTGAC
GCCGGACAAGGATCAGCTCTGCTCGTTGGTAAAGATTCGCCGGCTGCCG
AGCGACTCCATGCCCGTTGGGCTACTAGCCGTGTCAATAAGAGCATTAC
CCGCCTCCTCCGTCAGTTGGAGCTCCCTCCTACCGCCACGGCTACGGCC
TCGATAATGCCGCACGTGATGGAGCAGCCTCTCAGTACCAGCATTAACC
CCGTCAACGACCGCTTCAACGGTATTCCCAACCCCACTCCGTATAACTC
CGATGCAGCTCTCGATGCTATCACTCAGACCAACGATTATGGAAGCGTA
AATACACATGGTATCCTCTCTACTTACCCGCCACCGGCTACGCACCTTA
ATGAAGCTTCCGTCGCTCTCGCTCCCGGTGGCGCCCCCCCCCGACCGCC
TCCTCCGTATGTTGACAGCACGACCAATCACCCGCCGTACCACTCGAAT
CTGGTTCCAATGGCGAACTTTGGTTACTCGACCGTTGATTACGATGCCA
TGGTTGACGATTTGGCTAGCATTGAATACACGGACGCTGTGGATGTCGA
CCCACAGTTTATGACCAATCTGGGATTCGTTCCTGGATGTAACTTCTCC
GACATTAATACATACGAACAGTGA.

As used herein, the term “transgene” refers to a gene or genetic material that has been transferred or artificially introduced into the genome by a genetic engineering technique from one organism to another, i.e., the host organism.

As used herein, the term “transgene expression” relates to the control of the amount and timing of appearance of the functional product of a transgene in a host organism.

Inducible systems are inactive unless there is the presence of a molecule that allows for (induces) gene expression. In some aspects, placing the eukaryotic transcription factor binding site, QUAS, upstream of the promoter (without QF) very tightly represses gene expression of the transgene. Gene expression of the transgene can then be turned on with the addition of QF.

The term “sequence of interest” or “gene of interest” can mean a nucleic acid sequence (e.g., a therapeutic gene), that is partly or entirely heterologous, i.e., foreign, to a cell into which it is introduced.

The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be cDNA, DNA, or mRNA.

The term “sequence of interest” or “gene of interest” can also mean a nucleic acid sequence that is partly or entirely complementary to an endogenous gene of the cell into which it is introduced.

A “sequence of interest” or “gene of interest” can also include one or more transcriptional regulatory sequences and any other nucleic acid that may be necessary for optimal expression of a selected nucleic acid. A “protein of interest” means a peptide or polypeptide sequence (e.g., a therapeutic protein), that is expressed from a sequence of interest or gene of interest.

The term “endogenous” as used herein refers to substances and processes originating from within an organism, tissue or cell.

“Inhibit,” “inhibiting” and “inhibition” mean to diminish or decrease gene expression, activity, response, condition, disease, or other biological parameter. This can include, but is not limited to, the complete ablation of the activity, response, condition, or disease. This may also include, for example, a 10% inhibition or reduction in gene expression, activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

Although the foregoing invention has been described in some detail by way of illustration and example for purposes of clarity of understanding, certain changes and modifications may be practiced within the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

There is a growing need in the field of prokaryotic synthetic biology to develop next-generation genetic tools for various applications such as the construction of biosynthetic pathways for biofuel production, and the optimization of pathway flux for the production of biotherapeutic molecules. Given the complexity of cell signaling pathways, additional orthogonal genetic tools are needed to engineer more sophisticated control systems. Disclosed herein are compositions and methods for transferring eukaryotic genetic parts to prokaryotes demonstrating that gene expression functions from the bacteriophage T7 promoter RNA polymerase. Provided herein are compositions, methods and examples for adapting the activating eukaryotic transcription factor, QF, and its DNA binding domain (or sequence) to prokaryotes provides a means to introduce transcriptional activation, in addition to tight off states in Escherichia coli. These findings demonstrate a paradigm shift in prokaryotic synthetic biology by reversing the current approach of moving genetic parts between organisms where the prevailing trend is to move genetic parts from lower organisms to higher organisms. Described herein are methods of gene regulation systems for orthogonal genetic circuit design in prokaryotes.

Transcription factors (TFs) can be used to build genetic circuits to regulate gene expression. TFs are DNA binding proteins that repress or activate transcription once expressed from promoters. For example, in the natural system, the eukaryotic TF, QF, binds to its binding partner, QUAS, and controls the transcription of genes involved in quinic acid catabolism in the fungus Neurospora crassa (N. crassa). To activate gene expression, QF binds to its DNA binding sequence, QUAS, and activates transcription of catabolic enzymes (32-35). The N. crassa QUAS operator site for QF binding contains one or more copies of the 16-mer sequence GGGTAATCGCTTATCC (SEQ ID NO: 8). When quinic acid is present, QF activates expression of enzymes to catabolize quinic acid (N. H. Giles et al., Microbiol Rev 49, 338-358 (1985); and V. B. Patel, et al., Proceedings of the National Academy of Sciences of the United States of America 78, 5783-5787 (1981)). Previously, the QUAS operator sites and QF were engineered to enable transcriptional control in organisms including drosophila, zebrafish and mammalian cells (M. Fitzgerald, et al., ACS synthetic biology 6, 2014-2020 (2017); C. J. Potter, et al., Cell 141, 536-548 (2010); and X. Wei, et al., Nature methods 9, 391-395 (2012)). Placement of the 16 base pair QUAS operator sequence adjacent to the T7 promoter region permitted the investigation of the effect of QF binding on protein expression by the T7 system in E. coli. Bacteriophage T7 RNA polymerase (RNAP) is a single subunit polymerase that binds to the 17 base pair T7 promoter sequence with high specificity and is able to transcribe DNA in multiple organisms without additional protein subunits (D. J. Lee, et al., Annu Rev Microbiol 66, 125-152 (2012); and M. Chamberlin, et al., Nature 228, 227-231 (1970)). The T7 system is commonly used for high levels of orthogonal transcription in Escherichia coli (E. coli), where protein levels can reach 30% of total cell protein when expressed from a T7 promoter (F. W. Studier, et al., Methods Enzymol 185, 60-89 (1990)). The T7 RNAP does not bind native promoter sequences and has been used to control orthogonal transcription of genetic circuits in bacteria (B. A. Blount, et al., PloS one 7, e33279 (2012); H. Dong, et al., Journal of bacteriology. 177, 1497-1504 (1995); and W. An, J. W. Chin, Proceedings of the National Academy of Sciences of the United States of America 106, 8477-8482 (2009)).

Bacterial transcription occurs when endogenous multi-subunit bacterial RNA polymerase (RNAP) interacts with two DNA hexamer elements, −10 and −35, within the promoter sequence. In bacteria, activating TFs control endogenous RNAP recruitment by making contact with RNAP subunits upstream of the −35 element, in between the −35 and −10 elements or by altering the conformation of DNA at the promoter site (D. J. Lee, et al., Annu Rev Microbiol 66, 125-152 (2012)). Bacteriophage T7 RNAP is a single subunit polymerase that binds to the 17 base pair T7 promoter sequence with high specificity and is able to able to transcribe DNA in multiple organisms without additional protein subunits (M. Chamberlin, et al., Nature 228, 227-231 (1970); and F. W. Studier, et al., Methods Enzymol 185, 60-89 (1990)). The T7 system is commonly used for high levels of recombinant protein production in Escherichia coli (E. coli), where overexpressed protein levels can reach 30% of total cell protein (H. Dong, et al., Journal of bacteriology 177, 1497-1504 (1995)). The T7 RNAP does not bind native promoter sequences and has been used in synthetic biology to control orthogonal transcription of genetic circuits in bacteria (K. Temme, et al., Nucleic acids research 40, 8773-8781 (2012); and W. An, J. W. Chin, Proceedings of the National Academy of Sciences of the United States of America 106, 8477-8482 (2009)). Transcription factors modifying T7 RNAP transcription must bind to DNA upstream or downstream of the T7 promoter, not within the promoter as with bacterial RNAP (F. W. Studier, et al., Methods Enzymol 185, 60-89 (1990)). The most commonly used DNA binding sequences to control expression from a T7 promoter system is the lac operator (lacO). lacO sites enable repression of transcription by T7 RNAP with lad protein binding to the lacO site located two base pairs downstream of the T7 promoter (F. W. Studier, et al., Methods Enzymol 185, 60-89 (1990)). Controlling RNAP's ability to bind to DNA and initiate transcription is a classic approach to controlling gene expression in synthetic biology.

Synthetic biology tools include operator sites that control the repression of the T7 promoter using small molecules, most commonly the lacI and tetR systems (S. Auslander, M. Fussenegger, Trends in biotechnology 31, 155-168 (2013); W. Weber, M. Fussenegger, Curr Opin Chem Biol 15, 414-420 (2011); J. M. Callura, et al., Proceedings of the National Academy of Sciences of the United States of America 109, 5850-5855 (2012); and D. E. Cameron, et al., Nature reviews. Microbiology 12, 381-390 (2014)). To date, prokaryotic genetic modules, which are individual gene expression parts that can be independently characterized and used to build more sophisticated genetic tools, have been shown to function in eukaryotic systems (T. L. Deans, et al., Cell 130, 363-372 (2007); M. Fitzgerald, et al., ACS synthetic biology 6, 2014-2020 (2017); B. P. Kramer et al., Nature biotechnology 22, 867-870 (2004); M. Tigges, et al., Nature 457, 309-312 (2009); I. C. MacDonald, T. L. Deans, Adv Drug Deliv Rev 105, 20-34 (2016); and F. Lienert, et al., Nature reviews. Molecular cell biology 15, 95-107 (2014)). The ability to utilize eukaryotic transcriptional control systems in prokaryotes would allow orthogonal regulation of genetic circuits designed to implement cellular control. Disclosed herein are compositions, systems and methods that can reverse the current approach in synthetic biology in which genetic parts are moved from prokaryotes to higher organisms and demonstrate the utility of using eukaryotic genetic parts in prokaryotes to activate gene expression using bacteriophage RNAP. More specifically, the results described herein demonstrate the impact of moving the genetic regulatory parts from the eukaryote, N. crassa, to prokaryotes by placing the 16 base pair QUAS adjacent to a T7 promoter sequence in E. coli. The results also show that the eukaryotic transcription factor QF can be adapted to prokaryotes for improved orthogonal control of gene expression by preserving the protein's transcriptional activation abilities in prokaryotes.

### Binary Expression Systems

The ability to transfer eukaryotic genetic parts to prokaryotes to regulated gene and/or protein expression has the ability to revolutionize biology. Described herein are compositions and methods for regulating expression of an effector transgene or an endogenous gene using an inducible binary expression system. Using this strategy, transcriptional activation can be introduced by moving the activating eukaryotic transcription factor (e.g., QF) to a prokaryote. For example, a prokaryotic promoter (e.g., bacteriophage T7) can be positioned upstream from an endogenous operon (e.g., lac operon) that when activated can recruit an endogenous RNA polymerase. QF then binds to the eukaryotic transcription factor binding site sequence (e.g., QUAS operator), activating, for example, the QUAS promotor (e.g., T7) turning on gene (e.g., a gene of interest) expression. The eukaryotic transcription factor binding site sequence (e.g., QUAS operator, also referred to herein simply as “QUAS”) can have 5 QF binding sites. The QUAS operator allows for QF-dependent expression of the effector. In some aspects, a plasmid or an engineered construct can be used to position the T7 promoter into cell.

The compositions and methods described here can utilize regulatory genes from the Neurospora crassa qa gene cluster. This cluster consists of 5 structural genes and two regulatory genes (QA-1F and QA-1S) used for the catabolism of quinic acid as a carbon source (Giles et al., 1991). QA-1F (shortened as QF hereafter) is a transcriptional activator that binds to a 16-base pair sequence present in one or more copies upstream of each qa gene (Patel et al., 1981; Baum et al., 1987). QA-1S (shortened as QS hereafter) is a repressor of QF that blocks its transactivation activity (Huiet and Giles, 1986). Disclosed herein is the transfer of eukaryotic genetic parts to prokaryotes and demonstrating its utility to modulate gene expression in a prokaryotic cell system. In some aspects, one or more additional systems including but not limited to lad and tetR systems can be used in combination with or incorporated with or into the system described herein. In some aspects, a “system” utilizes genes from the qa cluster described herein.

Disclosed herein are inducible binary expression systems. In some aspects, the system can comprise: a) a first and second prokaryotic promoter; b) an operon, wherein the operon can be endogenous to a prokaryotic cell; c) a eukaryotic transcription factor, wherein the first prokaryotic promoter is operably linked to the eukaryotic transcription factor; d) a eukaryotic transcription factor binding site sequence, wherein the second prokaryotic promoter is operably linked to the eukaryotic transcription factor binding site sequence and wherein the eukaryotic transcription factor binding site sequence and the eukaryotic transcription factor are a binding pair; and e) one or more genes of interest. In some aspects, the one or more genes of interest can be regulated by the binding of the eukaryotic transcription factor to the eukaryotic transcription factor binding site sequence.

Disclosed herein are prokaryotic cells comprising the inducible expression. In some aspects, the system can comprise: a) a first and second prokaryotic promoter; b) an operon, wherein the operon is endogenous to a prokaryotic cell; c) an eukaryotic transcription factor, wherein the first prokaryotic promoter is operably linked to the eukaryotic transcription factor; d) a eukaryotic transcription factor binding site sequence, wherein the second prokaryotic promoter is operably linked to the eukaryotic transcription factor binding site sequence and wherein the eukaryotic transcription factor binding site sequence and the eukaryotic transcription factor are a binding pair; and e) one or more genes of interest. In some aspects, the one or more genes of interest can be regulated by the binding of the eukaryotic transcription factor to the eukaryotic transcription factor binding site sequence.

In some aspects, the first prokaryotic promoter can be located upstream of an operon. In some aspects, the operon can be endogenous to the one or more bacterial cells. In some aspects, the operon can be lac operon. In some aspects, the operon can be located between the prokaryotic promoter and the eukaryotic transcription factor. In some aspects, eukaryotic transcription factor binding site sequence can be located downstream of the first prokaryotic promoter. In some aspects, the eukaryotic transcription factor binding site sequence can be located upstream of the first prokaryotic promoter. In some aspects, the eukaryotic transcription factor binding site sequence can be located upstream of the second prokaryotic promoter. In some aspects, eukaryotic transcription factor binding site sequence can be located downstream of the eukaryotic transcription factor. In some aspects, the second prokaryotic promoter can be located upstream from one or more genes of interest or an effector gene. In some aspects, the second prokaryotic promoter can be located upstream from one or more genes of interest or an effector. In some aspects, the effector can be fluorescent proteins, ion channels, toxins and other genes. In some aspects, the second prokaryotic promoter can be located downstream of eukaryotic transcription factor binding site sequence.

Disclosed herein are prokaryotic cells. In some aspects, the prokaryotic cells can comprise: a first prokaryotic promoter operably linked to a eukaryotic transcription factor. In some aspects, the prokaryotic cell can comprise a prokaryotic promoter operably linked to a eukaryotic transcription factor. In some aspects, the eukaryotic transcription factor can be QF. In some aspects, the prokaryotic cells can comprise a first prokaryotic promoter operably linked to a eukaryotic transcription factor binding site sequence. In some aspects, the prokaryotic cell can comprise a prokaryotic promoter operably linked to a eukaryotic transcription factor binding site sequence. In some aspects, the eukaryotic transcription factor binding site sequence can be a QUAS sequence. In some aspects, the prokaryotic cell can comprising a first prokaryotic promoter operably linked to a eukaryotic transcription factor and a second prokaryotic promoter operably linked to a eukaryotic transcription factor binding site sequence. In some aspects, the prokaryotic cell can comprise a first prokaryotic promoter operably linked to a eukaryotic transcription factor, and wherein the prokaryotic cell comprises a construct, wherein the construct comprises a second prokaryotic promoter operably linked to a eukaryotic transcription factor binding site sequence and one or more genes of interest. In some aspects, the prokaryotic cell can comprise a second prokaryotic promoter operably linked to a eukaryotic transcription factor binding site sequence and a gene of interest, and wherein the prokaryotic cell comprises a construct, wherein the construct comprises a first prokaryotic promoter operably linked to a eukaryotic transcription factor. In some aspects, the eukaryotic transcription factor can be QF and the eukaryotic transcription factor binding site sequence can be a QUAS sequence. In some aspects, eukaryotic transcription factor binding site sequence can be located upstream of the second prokaryotic promoter.

In some aspects, the prokaryotic cell can comprise a construct. In some aspects, the construct can comprise a second prokaryotic promoter operably linked to a eukaryotic transcription factor binding site sequence and one or more genes of interest. In some aspects, the eukaryotic transcription factor binding site sequence can be QUAS. In some aspects, the prokaryotic cells can comprise a second prokaryotic promoter operably linked to a eukaryotic transcription factor binding site sequence and a gene of interest. In some aspects, the prokaryotic cell can comprise a construct. In some aspects, the construct can comprise a first prokaryotic promoter operably linked to a eukaryotic transcription factor. In some aspects, the prokaryotic cell can comprise: a) a first construct and a second construct. In some aspects, the first construct can comprise a first prokaryotic promoter operably linked to a eukaryotic transcription factor. In some aspects, the second construct can comprise a second prokaryotic promoter operably linked to a eukaryotic transcription factor binding site sequence and one or more genes of interest. In some aspects, the eukaryotic transcription factor can be QF and the eukaryotic transcription factor binding site sequence can be a QUAS sequence. In some aspects, eukaryotic transcription factor binding site sequence can be located upstream of the second prokaryotic promoter.

Disclosed herein are prokaryotic cells comprising a first construct, wherein the first construct comprises a first prokaryotic promoter operably linked to a eukaryotic transcription factor, and a second construct, wherein the second construct comprises a second prokaryotic promoter operably linked to a eukaryotic transcription factor binding site sequence and one or more genes of interest. In some aspects, the eukaryotic transcription factor can be QF. In some aspects, the eukaryotic transcription factor binding site sequence can be QUAS sequence. In some aspects, the eukaryotic transcription factor can be QF and the eukaryotic transcription factor binding site sequence be a QUAS sequence.

Any of the cells disclosed herein can be screened for expression (over or under expression) of one or more effectors, transgenes or one or more genes of interest.

Promoters. In some aspects, the prokaryotic promoter can be T7. Other bacteriophage promoters can be used. Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s). The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

In some aspects, the prokaryotic promoter can be T7. In some aspects, the prokaryotic promoter can be a bacterial promoter. In some aspects, the prokaryotic promoter can be an exogenous promotor that can be integrated into the genome of the bacterial cell that is being transformed. In some aspects, the prokaryotic promoter in the first and second construct can be T7. Examples of prokaryotic promoters that can be used in the compositions or methods disclosed herein include, but are not limited to, T7, T7lac, Sp6, araBAD, trp, lac, Ptac, and pL.

Transcription factors. Generally, transcription factors can work alone or with other proteins in a complex, by promoting (as an activator) or blocking (as a repressor) the recruitment of RNA polymerase (the enzyme that performs the transcription of genetic information from DNA to RNA) to one or more genes to ultimately regulate the expression or repression of one or more genes. Any suitable eukaryotic transcription factor can be used. In some aspects, any eukaryotic transcription factor and its respective binding site can be used. In some aspects, the eukaryotic transcription factor can be QF. In some aspects, the eukaryotic transcription factor binding site sequence and the eukaryotic transcription factor are a binding pair. In some aspects, the eukaryotic transcription factor can be QF and the eukaryotic transcription factor binding site sequence can be a QUAS sequence. Examples of transcription factors that can be used in the compositions or methods disclosed herein include, but are not limited to, are SP1, AP-1, C/EBP, heat shock factor, ATF/CREB, c-Myc, Oct-1, NF-1, GAL4 system, and estrogen receptor system (ER2).

Cell types. A host cell can be selected depending on the nature of the transfection vector, plasmid or construct. In some aspects, the host cell can be a prokaryotic cell. In some aspects, the host cell can be one or more bacterial cells. The cell can be examined using a variety of different physiologic assays. Such assays and methods are known to one skilled in the art. In some aspects, any cell can be a host cell. In some aspects, the host cell can be a mammalian cell. In some aspects, the host cell can be a cell line. Examples of prokaryotic cells types that can be used in the compositions or methods disclosed herein include, but are not limited to, to E. coli, Streptococcus bacterium, and archaea.

Constructs. The term “construct” as used herein can refer to a nucleic acid construct. The construct can be produced either through recombinant techniques or synthetically that will result in the transcription of a certain polynucleotide sequence in a host cell. The construct can be part of a plasmid, viral genome or nucleic acid fragment. Generally, the construct includes a polynucleotide operably linked to a promoter. In some aspects, a construct comprises a first and or second promoter. In some aspects, the construct can be in a plasmid. In some aspects, the construct can be in a single plasmid. In some aspects, the construct can be in two or more plasmids. The constructs can be adapted for expression in a specific type of host cell (e.g., using a specific type of promoter). The constructs can also comprise other components such as eukaryotic transcription factors or eukaryotic transcription factor binding site sequences or any other component that results in the expression of an effector or one or more genes of interest as disclosed herein in one or more bacterial cells.

As used herein, the term “operably linked” refers to the position of a regulatory region and a sequence to be transcribed in a nucleic acid to facilitate transcription or translation of the sequence. The choice of promoters depends on several factors including but not limited to efficiency, selectability, inducibility, desired expression level, and cell- or tissue-preferential expression. One skilled in the art is capable of appropriately selecting and positioning promoters and other regulatory regions relative to the coding sequence. In some aspects, operably linked can refer to where the regulatory region is upstream (i.e. 5′) of a nucleic acid sequence (e.g., a eukaryotic transcription binding sequence). In some aspects, operably linked can refer to where the regulatory region is downstream (i.e., 3′) of a nucleic acid sequence (e.g. a eukaryotic transcription binding sequence).

Vectors. Vectors comprising any of the constructs as described herein are also provided. As used herein, a “vector” refers a carrier molecule into which another DNA segment can be inserted to initiate replication of the inserted segment. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polyadenylation signal). Examples of vectors that are routinely used in the art include plasmids and viruses. The term “vector” includes expression vectors and refers to a vector containing a nucleic acid sequence coding for at least part of a gene product capable of being transcribed. A variety of ways can be used to introduce an expression vector into cells. In some aspects, the expression vector comprises a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from, for example, bacteriophages, retroviruses (e.g., lentiviruses), and other viruses (e.g., adenoviruses, poxviruses, herpesviruses and adeno-associated viruses). Vectors and expression systems are commercially available and known to one skilled in the art.

The vectors disclosed herein can also include detectable labels. Such detectable labels can include a tag sequence designed for detection (e.g., purification or localization) of an expressed polypeptide. Tag sequences include, for example, green fluorescent protein, glutathione S-transferase, polyhistidine, c-myc, hemagglutinin, or Flag™ tag, and can be fused with the encoded polypeptide and inserted anywhere within the polypeptide, including at either the carboxyl or amino terminus.

Increase gene expression. One or more of the constructs disclosed herein can be used to induce, promote or increase gene expression of one or more genes of interest. The constructs disclosed herein can comprise two different T7 promoters. For example, expression of QF can be controlled by a first T7 promoter that can be operably linked to a lac operon that can be endogenous to a bacterial cell (e.g., host cell). Expression of the gene of interest can be driven, for example, by the QUAS sequence that can be operably linked to a second promoter T7 promoter. When QF is absent, gene expression that can be driven by the QUAS sequence that can be operably linked to a second T7 promoter that is in the off state. When expression of QF is activated by the first T7 promoter which in turn drives lac operon to recruit an RNA polymerase, which in turn leads to the binding of the eukaryotic QF to bind to the QUAS sequence thereby drives the second T7 promoter to increase (or turn on) gene expression. In some aspects, the operon can be lac operon. In some aspects, the endogenous operon can be located between the first prokaryotic promoter (e.g., T7) and the eukaryotic transcription factor (e.g., QF). In some aspects, the QUAS sequence can be located or positioned upstream or downstream of the second prokaryotic promoter.

### Methods

Disclosed herein are methods of controlling expression of one or more genes of interest in one or more bacterial cells. In some aspects, the methods can comprise transforming the cells with one or more constructs. In some aspects, the method can comprise: a) transforming the cells with a first construct. In some aspects, the first construct can comprise a first prokaryotic promoter operably linked to a eukaryotic transcription factor. In some aspects, the method can comprise: b) transforming the cells with a second construct. In some aspects, the second construct can comprise a second prokaryotic promoter operably linked to a eukaryotic transcription factor binding site sequence and one or more genes of interest. In some aspects, the eukaryotic transcription factor binding site sequence and the eukaryotic transcription factor can be a binding pair. In some aspects, expression of the one or more genes of interest in said one or more bacterial cells can be regulated by binding of said eukaryotic transcription factor to the eukaryotic transcription factor binding site sequence. In some aspects, the eukaryotic transcription factor binding site sequence can be located upstream of the second prokaryotic promoter. In some aspects, the eukaryotic transcription factor binding site sequence can be located downstream of the second prokaryotic promoter. In some aspects, the compositions and methods described herein can include or use a single construct such that one or more genes of interest in a single bacterial cell (e.g., a single plasmid) can be regulated. In some aspects, the compositions and methods described herein can include more than one construct such that one or more genes of interest in a single bacterial cell (e.g., a single plasmid) can be regulated.

Disclosed herein are methods of controlling expression of one or more genes of interest in one or more bacterial cells. In some aspects, the method can comprise transforming the cells with a first construct. In some aspects, the first construct can comprise a first prokaryotic promoter operably linked to a eukaryotic transcription factor. In some aspects, the one or more bacterial cells can comprise a second construct. In some aspects, the second construct can comprise a second prokaryotic promoter operably linked to a eukaryotic transcription factor binding site sequence and one or more genes of interest. In some aspects, the eukaryotic transcription factor binding site sequence and the eukaryotic transcription factor can be a binding pair. In some aspects, expression of the one or more genes of interest in said one or more bacterial cells can be regulated by binding of said eukaryotic transcription factor to the eukaryotic transcription factor binding site sequence. In some aspects, the eukaryotic transcription factor binding site sequence can be located upstream of the second prokaryotic promoter. In some aspects, the eukaryotic transcription factor binding site sequence can be located downstream of the second prokaryotic promoter.

Disclosed herein are methods of controlling expression of one or more genes of interest in one or more bacterial cells. In some aspects, the method can comprise transforming the cells with a second construct. In some aspects, the second construct can comprise a second prokaryotic promoter operably linked to a eukaryotic transcription factor binding site sequence and a gene of interest. In some aspects, the one or more bacterial cells can comprise a first construct. In some aspects, the first construct can comprise a first prokaryotic promoter operably linked to a eukaryotic transcription factor. In some aspects, expression of the one or more genes of interest in said one or more bacterial cells can be regulated by binding of said eukaryotic transcription factor to the eukaryotic transcription factor binding site sequence. In some aspects, the eukaryotic transcription factor binding site sequence can be located upstream of the second prokaryotic promoter. In some aspects, the eukaryotic transcription factor binding site sequence can be located downstream of the second prokaryotic promoter.

In some aspects, the one or more bacterial cells can be E. coli. In some aspects, the bacterial cell can be any suitable bacterial cell. In some aspects, the one or more bacterial cells can be a Streptococcus bacterium. In some aspects, the one or more bacterial cells can be archaea.

In some aspects, the one or more genes of interest can be an effector transgene. In some aspects, the method can further comprise transforming the cells with the effector transgene, whereby expression of said effector transgene is regulated by binding of said eukaryotic transcription factor to said eukaryotic transcription factor binding site sequence.

In some aspects, the eukaryotic transcription factor binding site sequence can be a QUAS sequence. In some aspects, the eukaryotic transcription factor can be QF. Examples of transcription factors that can be used in the compositions or methods disclosed herein include, but are not limited to, SP1, AP-1, C/EBP, heat shock factor, ATF/CREB, c-Myc, Oct-1, NF-1, GAL4 system, and estrogen receptor system (ER2). In some aspects, the eukaryotic transcription factor binding site sequence and the eukaryotic transcription factor are a binding pair. In some aspects, the eukaryotic transcription factor can be QF and the eukaryotic transcription factor binding site sequence can be a QUAS sequence.

In some aspects, the prokaryotic promoter can be T7. In some aspects, the prokaryotic promoter can be a bacterial promoter. In some aspects, the prokaryotic promoter can be an exogenous promotor that can be integrated into the genome of the bacterial cell that is being transformed. In some aspects, the prokaryotic promoter in the first and second construct can be T7.

In some aspects, the first prokaryotic promoter can be located upstream of an operon. In some aspects, the operon can be endogenous to the one or more bacterial cells. In some aspects, the operon can be lac operon. In some aspects, the operon can be located between the prokaryotic promoter and the eukaryotic transcription factor. In some aspects, eukaryotic transcription factor binding site sequence can be located downstream of the first prokaryotic promoter. In some aspects, the eukaryotic transcription factor binding site sequence can be located upstream or downstream of the first prokaryotic promoter. In some aspects, eukaryotic transcription factor binding site sequence can be located downstream of the eukaryotic transcription factor. In some aspects, the second prokaryotic promoter can be located upstream from one or more genes of interest or an effector gene. In some aspects, the second prokaryotic promoter can be located upstream from one or more genes of interest or an effector. In some aspects, the effector can be fluorescent proteins, ion channels, toxins and other genes. In some aspects, the second prokaryotic promoter can be located downstream of eukaryotic transcription factor binding site sequence.

In some aspects, the expression of one or more genes of interest can be increased.

### EXAMPLES

### Example 1: Prokaryotic Gene Expression Regulated by a Eukaryotic Transcription Factor

Genetic tools that artificially control gene expression can be used to study gene function and construct gene networks to reprogram cells with desired properties. Described herein are experiments that move genetic parts from lower organisms to higher organisms, demonstrating that eukaryotic genetic parts can be moved to prokaryotes to introduce transcriptional activation, in addition to tight off states. Altogether, this studies provides evidence for drastically increasing the number of orthogonal genetic tools to be used in prokaryotic synthetic biology, and improve recombinant protein production techniques.

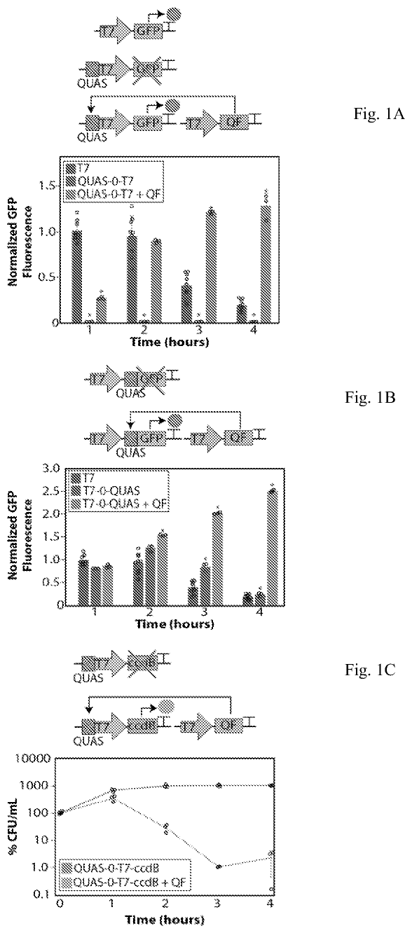

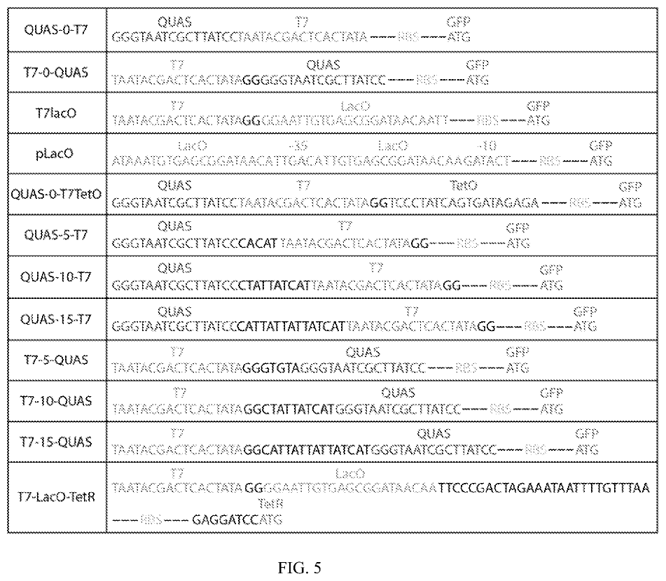

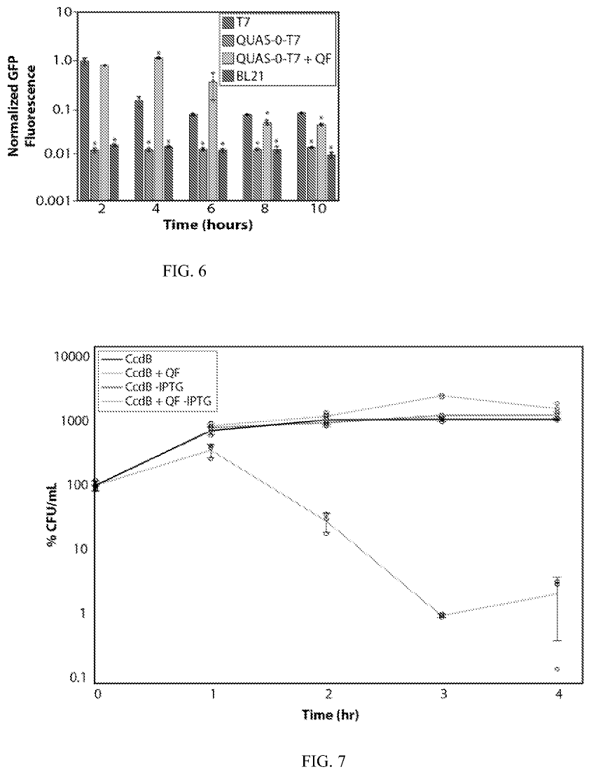

QUAS upstream of T7 enables off/on gene expression. To investigate the effect of the eukaryotic transcription factor QF on prokaryotic gene expression, the 16 base pair QUAS was placed directly upstream of the T7 promoter (QUAST7) driving the expression of green fluorescent protein (GFP) (FIG. 1A, FIG. 5). A degradation tag was added to the C-terminal end of GFP to investigate the expression dynamics from the tested constructs (J. B. Andersen et al., Appl Environ Microbiol 64, 2240-2246 (1998); K. E. McGinness, et al., Molecular cell 22, 701-707 (2006); and J. H. Davis, et al., ACS chemical biology 6, 1205-1213 (2011)). In order to contextualize data from this system, the T7 promoter was followed by a lacO operator site, which served as the T7 promoter control driving GFP expression. Fluorescence values measured by flow cytometry show that placement of the QUAS upstream of the T7 promoter held GFP expression off in the absence of QF when compared to the T7 promoter control (FIG. 1B), and had fluorescence equivalent to the background fluorescence level of untransformed cells (FIG. 6). When QF was expressed in the QUAST7 system, QF bound to QUAS and activated the expression of GFP from QUAST7 (FIG. 1B). The GFP fluorescence driven by QUAST7 when QF was present surpassed the level from the T7 promoter control after two hours (FIG. 1B). To investigate how long this higher level of expression was maintained, GFP expression was observed over 10 hours (FIG. 1C). QUAST7 produced switch behavior dependent on QF with a tight off state and robust activation of GFP expression.

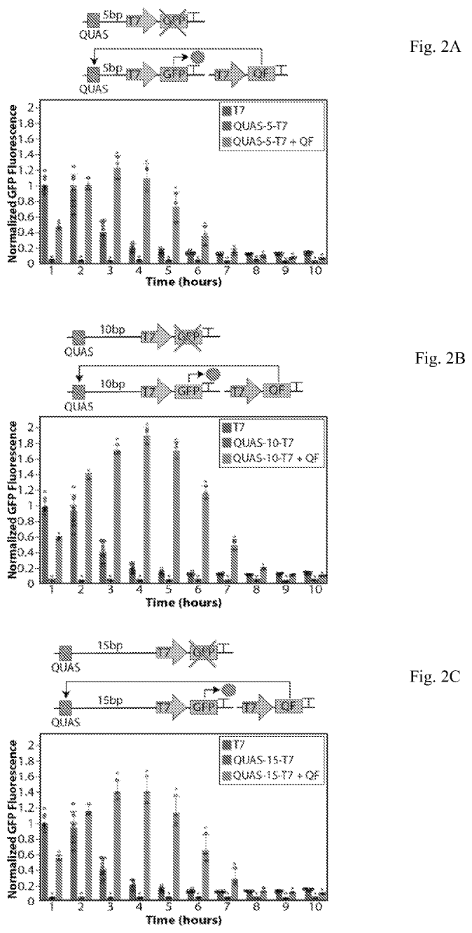

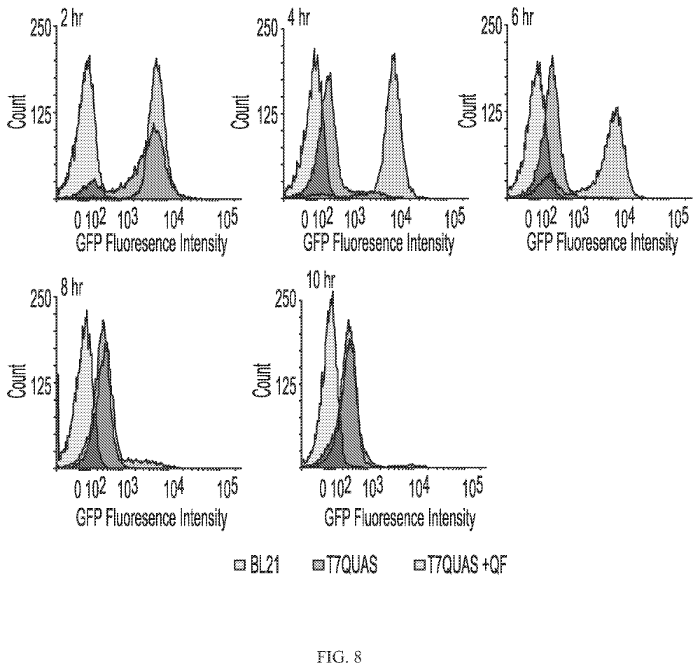

Engineering a CcdB kill switch. To more clearly demonstrate the influence of placing QUAS upstream of the T7 promoter, and to highlight the level of gene silencing achieved by this eukaryotic DNA binding sequence in prokaryotes, GFP was replaced with CcdB (FIG. 2A). CcdB is the toxic protein in the CcdB/CcdA toxin/antitoxin system that causes severe DNA damage and cell death (E. M. Bahassi et al., The Journal of biological chemistry 274, 10936-10944 (1999); H. Afif, et al., Molecular microbiology 41, 73-82 (2001); J. M. Callura, et al., Proceedings of the National Academy of Sciences of the United States of America 107, 15898-15903 (2010); D. J. Dwyer, et al., Annu Rev Pharmacol Toxicol 55, 313-332 (2015); and D. J. Dwyer, et al., Molecular systems biology 3, 91 (2007)). E. coli cells with QUAST7 regulating the transcription of CcdB were able to grow over a 4 hour time period without growth inhibition or cell death, due to the tight off state that the QUAS provides (FIG. 2B and FIG. 7). The expression of QF activated the CcdB expression from QUAST7 and caused 99% cell death within two hours of induction (FIG. 2B), demonstrating that the placement of the eukaryotic QUAS upstream of the T7 promoter can be used to effectively turn any gene off and, with QF, on in prokaryotes.

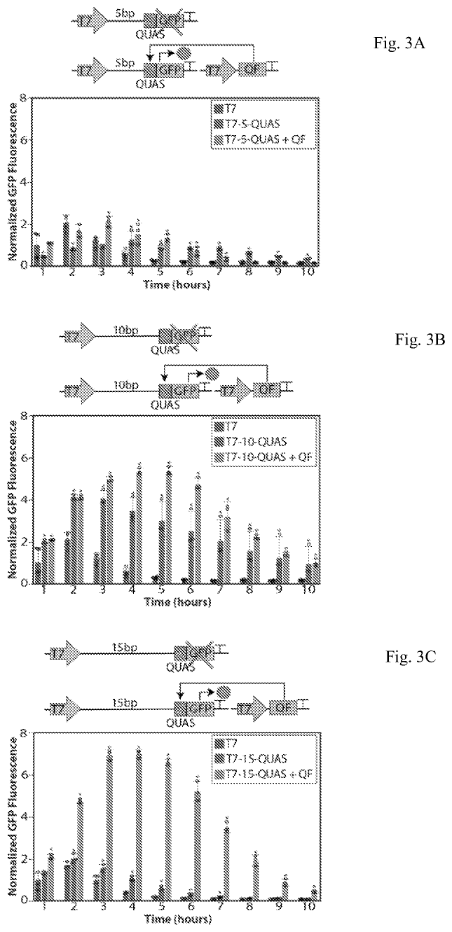

QUAS spacing upstream of the T7 promoter impacts gene expression. To determine whether the location of the QUAS influenced GFP gene expression, the QUAS was moved upstream of the T7 promoter. Because one half-turn of the DNA double-helix is approximately 5 base pairs, the placement of QUAS was shifted in intervals of 5 base pairs, enabling the bound QF orientation to be rotated one half-turn (FIG. 3). QUAS was therefore placed 5, 10, or 15 base pairs upstream of the T7 promoter (FIG. 3). The three QUAS spacing plasmids produced switch-like expression with tight off states in the absence of the QF transcription factor, and activation of gene expression in the presence of QF. Of the positions tested, the QUAS placed 10 base pairs upstream of the T7 promoter (QUAS10T7) produced the highest expression of GFP when QF was present (FIG. 3B). Additionally, QUAS10T7 with QF maintained a robust level of expression for a longer period of time when compared to all other upstream locations.

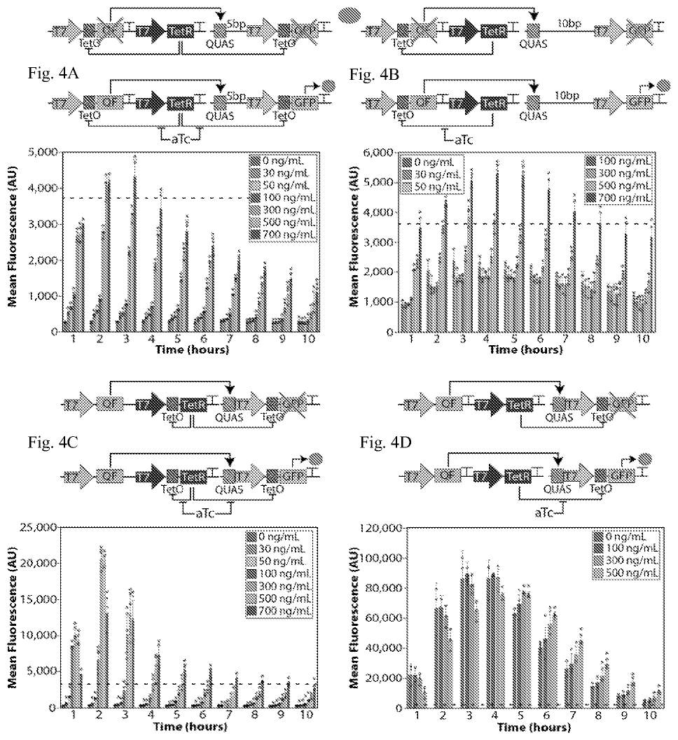

QUAS downstream of T7 promoter enables amplification of gene expression. Next, the DNA binding sequence QUAS was placed downstream of the T7 promoter (T7QUAS) (FIG. 4A). In the absence of QF, GFP expression from T7QUAS was similar to the expression of GFP from the T7 control (FIG. 4B). The expression of GFP in both the T7 control and T7QUAS without QF peaked at 2 hours post induction. However, expression quickly diminished by 3 hours from both the T7 control and T7QUAS without QF.

In contrast, GFP expression was amplified when QF bound to the QUAS downstream of T7, with fluorescence values greater than the T7 control. This expression increase was prolonged for 6 hours, expression lasting 4 hours longer than T7QUAS and the T7 control (FIG. 4C). Over the time-course of the experiment, T7QUAS with QF was capable of keeping more of the bacterial population expressing GFP than the T7 control, in addition to maintaining higher levels of GFP expression per cell compared to the T7 control cells (FIG. 8). Altogether, placing the QUAS downstream of the T7 promoter and expressing the eukaryotic QF transcription factor enabled higher GFP expression levels, and GFP expression for a longer duration when compared to the T7 control (FIG. 4D).

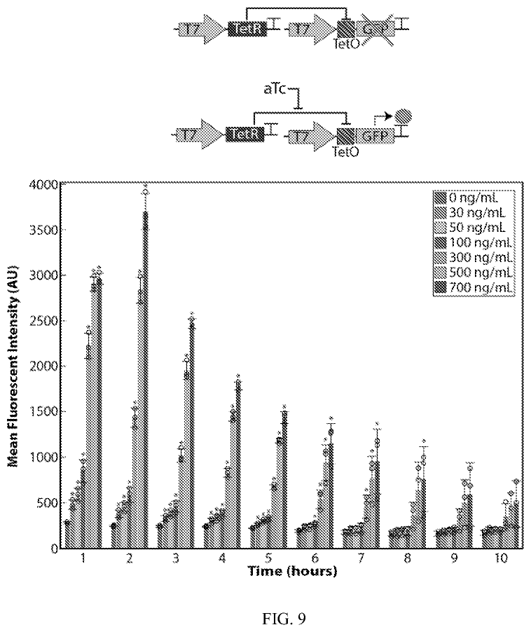

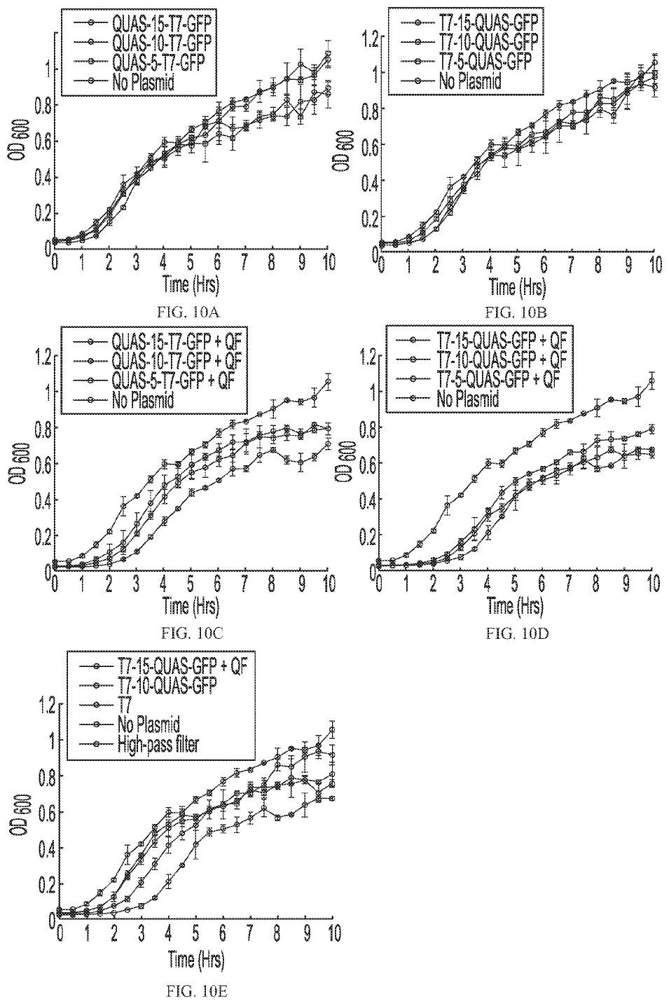

To examine whether transcriptional activation by QF relies on binding DNA near the T7 promotor, the DNA binding domain from QF was removed and the QF activation domain (QF_{AD}) was expressed. In this case, GFP expression from T7QUAS was not increased in the presence of QF_{AD} (FIG. 9). For both the T7 control and T7QUAS, the level of GFP expression decreased with QF_{AD} in the system. This decrease is likely because QF_{AD} expression was driven by a T7 promotor which recruits T7 RNAP and other resources away from GFP expression (T. H. Segall-Shapiro, et al., Molecular systems biology 10, 742 (2014); D. Das, et al., PLoS computational biology 13, e1005491 (2017); and J. Landman, et al., PloS one 12, e0179235 (2017)). The inability of QF_{AD} to increase GFP expression demonstrates a reliance on the DNA binding domain of QF to localize the transcription factor to where the activation domain can interact favorably with T7 RNAP.

Discussion. Synthetic biology tools employ operator sites and transcription factors that control the repression of transcription by RNAP using small molecules, most commonly the lad and tetR systems (S. Auslander, M. Fussenegger, Trends in biotechnology 31, 155-168 (2013); W. Weber, M. Fussenegger, Curr Opin Chem Biol 15, 414-420 (2011); J. M. Callura, et al., Proceedings of the National Academy of Sciences of the United States of America 109, 5850-5855 (2012); and D. E. Cameron, et al., Nature reviews. Microbiology 12, 381-390 (2014)). To date, prokaryotic genetic modules have been shown to function in eukaryotic systems (T. L. Deans, et al., Cell 130, 363-372 (2007); M. Fitzgerald, et al., ACS synthetic biology 6, 2014-2020 (2017); B. P. Kramer et al., Nature biotechnology 22, 867-870 (2004); M. Tigges, et al., Nature 457, 309-312 (2009); I. C. MacDonald, T. L. Deans, Adv Drug Deliv Rev 105, 20-34 (2016); and F. Lienert, et al., Nature reviews. Molecular cell biology 15, 95-107 (2014), however, it has not been shown whether eukaryotic transcription factors can carry over their activation into prokaryotic systems. The ability to utilize eukaryotic transcriptional control systems in prokaryotes will permit expanded orthogonal regulation of genetic circuits designed to implement complex cellular gene control.

The eukaryotic transcription factor, QF, along with its binding site, QUAS, demonstrates control over uncommon cellular behaviors when adapted to T7 expression in E. coli. Placing the QUAS upstream of the T7 promoter permits a tight off state of expression in the absence of QF, and GFP expression is activated when QF is present in the system. It was tested whether the QUAST7 spacings were held in the tight off state in the absence of QF by non-specific endogenous protein binding that represses transcription by T7 RNAP, however, when QF is present, GFP is expressed from QUAST7 (FIG. 1B). Favorable interaction dynamics between QF and RNAP are capable of improving the expression level and duration. The impact of spacing on the activating function of QF indicates potential for a orientation and location of QF binding to be determined with an expanded spacing library.

Moving the DNA binding sequence QUAS downstream of the T7 promoter permits strong amplification of gene expression in the presence of QF. Expression levels from T7QUAS with QF were amplified beyond the levels of expression by the T7 control and any of the T7 promoters with an upstream QUAS. The ability of QF to bind to the QUAS oriented on either side of the T7 promoter to produce vastly different expression profiles demonstrates the potential of QF and related eukaryotic transcription factors to control gene expression through mechanisms unseen in bacteria to date. The mechanism of transcriptional control by the QUAS and QF, while yet to be determined, is uncommon compared to previously developed bacterial activator systems because it controls expression from an orthogonal RNAP rather than from the native RNAP.

An activator for the T7 expression system is new, and this ON/OFF control of gene expression at the T7 promoter represents a method to expand the control over the T7 expression system in the bacterial synthetic biology toolbox. The data described herein show that bringing a transcription factor from a higher organism into a bacterial expression system using bacteriophage RNAP permits new control mechanisms, including programmed activation in prokaryotes that can be used to advance prokaryotic synthetic biology and biotechnology applications. This work has the potential to transform the field of synthetic biology by providing a new regulation strategy for genetic circuit design and represents a paradigm shift by transferring eukaryotic transcription factors to prokaryotic hosts.

### Materials and Methods

QUAS operator T7 promoter construction. A single 16-mer QUAS (GGGTAATCGCTTATCC; SEQ ID NO: 8) was oriented upstream or downstream of the T7 promoter sequence. To clone the QUAST7 and T7QUAS promoter-operators, DNA oligos were annealed and then ligated into a vector with ampicillin resistance and a ColEI origin of replication. To maintain transcription in the T7QUAS expression system, the +1 and +2 base pairs were conserved (GG) and QUAS was inserted after these two base pairs (T7QUAS) (FIG. 5). QUAS5T7, QUAS10T7, and QUAS15T7 were cloned using geneblocks (IDT). These double stranded oligos were digested and ligated into the p15A and ampicillin resistance expression backbone containing GFP with a degradation tag. The degradation tag sequence (DAS+4) was chosen to leverage bacterial ssra degradation machinery to allow for the rapid degradation of GFP and CcdB (K. E. McGinness, et al., Molecular cell 22, 701-707 (2006)). The QF sequence was PCR amplified from pAC-7-QFBDAD (Addgene plasmid #46096) (O. Riabinina et al., Nature methods 12, 219-222, 215 p following 222 (2015)) and restriction enzyme cloned downstream of the T7lacO promoter in a vector backbone with kanamycin resistance and p15A origin of replication. The QUAST7 promoter driving CcdB expression vector was cloned by PCR amplifying CcdB with the degradation tag and restriction enzyme cloning downstream of the QUAST7 promoter in place of GFP. All plasmid transformations used DH5α chemically competent cells (ThermoFisher). GFP expression experiments. GFP expression experiments were conducted with chemically competent BL21 DE3 E. coli (ThermoFisher), engineered to express T7 RNAP when induced with isopropyl β-D-1-thiogalactopyranoside (IPTG). QUAST7 and T7QUAS expression experiments without QF were conducted by transforming the GFP expression plasmid alone into the BL21 cells. QUAST7 and T7QUAS expression experiments with QF were conducted by co-transforming the GFP expression plasmid and the QF expression plasmid into BL21. GFP expression plasmids contained ampicillin resistance and a ColEI origin of replication. QF expression plasmid contained kanamycin resistance and a p15A origin of replication. A single transformed BL21 colony of was picked and grown overnight in LB (Fisher Scientific) with selection at 37° C. and shaking at 280 RPM. The overnight culture was diluted 1:50 in LB with selection and grown up at 37° C. and shaken at 280 RPM. At OD600 (Synergy HTX Reader, Biotek) of ˜0.2, cultures were induced with 0.5 mM IPTG. Induction served as the time zero point for time point experiments.

Flow Cytometry. Samples were diluted 1:1000 in PBS and analyzed on a DXP flow cytometer at time points 1-10 hours post induction with IPTG. GFP expression was measured using a 488 nm laser and a 530/30 filter. Populations of 10,000 cells were used to calculate GFP fluorescence. Flow data was analyzed using Flowing Software (Cell Imaging Core, Turkey Centre for Biotech). GFP fluorescence data in FIGS. 1, 3 and 4 were normalized by dividing the mean fluorescence of each sample by the mean of GFP fluorescence from the T7 control at after 1 hour of induction.

CcdB expression experiments. QUAST7 expression experiments without QF were conducted by transforming the expression plasmid alone into BL21 DE3 cells. QUAST7 with QF experiments were conducted by co-transforming the expression plasmid with the QF plasmid into BL21 DE3 cells. A single transformed BL21 colony was picked and grown overnight in LB (Fisher Scientific) with antibiotic selection at 37° C. and shaken at 280 RPM. The overnight culture was diluted 1:50 in LB with selection and grown up at 37° C. and shaken at 280 RPM. At OD600 (Synergy HTX Reader, Biotek) of ˜0.2 cultures were induced with 10 μM IPTG. Induction with IPTG was done at the time (T) zero point for time point experiments. At T=0-4 hours, samples were diluted 1:10 consecutively in PBS and plated on LB agar plates with antibiotic selection. These plates were placed in a 37° C. incubator overnight, and colonies were counted the next day to calculate colony forming units (CFUs).

QF_{AD}. The DNA binding domain and activation domain of QF have been previously studied (O. Riabinina et al., Nature methods 12, 219-222, 215 p following 222 (2015)). The activation domain was PCR amplified from the QF expression plasmid and cloned into a vector with kanamycin resistance, p15A origin and T7lac0 driving QF_{AD}. QF_{AD} was co-transformed alongside T7QUAS. Methods for growth, induction and flowcytometry follow the steps outlined herein.

1. A method of controlling expression of one or more genes of interest in one or more bacterial cells, the method comprising:
a) transforming the cells with a first construct, wherein the first construct comprises a first prokaryotic promoter operably linked to a eukaryotic transcription factor, and
b) transforming the cells with a second construct, wherein the second construct comprises a second prokaryotic promoter operably linked to a eukaryotic transcription factor binding site sequence and one or more genes of interest, wherein the eukaryotic transcription factor binding site sequence and the eukaryotic transcription factor are a binding pair,
whereby expression of the one or more genes of interest in said one or more bacterial cells is regulated by binding of said eukaryotic transcription factor to the eukaryotic transcription factor binding site sequence.

2. A method of controlling expression of one or more genes of interest in one or more bacterial cells, the method comprising:
transforming the cells with a first construct, wherein the first construct comprises a first prokaryotic promoter operably linked to a eukaryotic transcription factor, and wherein said cells comprise a second construct, wherein the second construct comprises a second prokaryotic promoter operably linked to a eukaryotic transcription factor binding site sequence and one or more genes of interest, wherein the eukaryotic transcription factor binding site sequence and the eukaryotic transcription factor are a binding pair,
whereby expression of the one or more genes of interest in said one or more bacterial cells is regulated by binding of said eukaryotic transcription factor to the eukaryotic transcription factor binding site sequence.

3. (canceled)

4. The method of claim 1, wherein the eukaryotic transcription factor is QF.

5. The method of claim 1, wherein the eukaryotic transcription factor binding site sequence is a QUAS sequence.

6. The method of claim 1, wherein the eukaryotic transcription factor is QF and the eukaryotic transcription factor binding site sequence is a QUAS sequence.

7. The method of claim 1, wherein the one or more genes of interest is an effector transgene, and the method further comprises:
transforming the cells with the effector transgene, whereby expression of said effector transgene is regulated by binding of said eukaryotic transcription factor to said eukaryotic transcription factor binding site sequence.

8. The method of claim 1, wherein the prokaryotic promoter in the first and second construct is T7.

9. The method of claim 1, wherein the prokaryotic promoter of the first construct is located upstream of an operon, wherein the operon is endogenous to the one or more bacterial cells.

10. The method of claim 9, wherein the operon is lac operon.

11. The method of claim 10, wherein the operon is located between the prokaryotic promoter and the eukaryotic transcription factor.

12. The method of claim 1, wherein the expression of one or more genes of interest is increased.

13. An inducible binary expression system, the system comprising:
a) a first and second prokaryotic promoter;
b) an operon, wherein the operon is endogenous to a prokaryotic cell;
c) a eukaryotic transcription factor, wherein the first prokaryotic promoter is operably linked to the eukaryotic transcription factor;
d) a eukaryotic transcription factor binding site sequence, wherein the second prokaryotic promoter is operably linked to the eukaryotic transcription factor binding site sequence and wherein the eukaryotic transcription factor binding site sequence and the eukaryotic transcription factor are a binding pair; and
e) one or more genes of interest, wherein the one or more genes of interest is regulated by the binding of the eukaryotic transcription factor to the eukaryotic transcription factor binding site sequence.

14. The inducible binary expression system of claim 13, wherein the eukaryotic transcription factor is QF.

15. The inducible binary expression system of claim 13, wherein the eukaryotic transcription factor binding site sequence is a QUAS sequence.

16. The inducible binary expression system of claim 13, wherein the eukaryotic transcription factor is QF and the eukaryotic transcription factor binding site sequence is a QUAS sequence.

17. The inducible binary expression system of claim 13, wherein the first and second prokaryotic promoter is T7.

18. The inducible binary expression system of claim 13, wherein the operon is lac operon.

19. The inducible binary expression system of claim 13, wherein the endogenous operon is located between the prokaryotic promoter and the eukaryotic transcription factor.

20. A prokaryotic cell comprising the inducible binary expression system of claim 13.

21.-40. (canceled)
